(19) United States
(12) Patent Application Publication
(10) Pub. No.: US 2023/0170066 A1
(43) Pub. Date: Jun. 1, 2023

(54) DIGITIZED DATA INPUT DEVICE AND METHOD

(71) Applicant: ADoH SCIENTIFIC, LLC, Mt. Pleasant, SC (US)

(72) Inventors: BRIAN SULLIVAN, CHARLESTON, SC (US); ALFRED FASOLA, JR., SUMMERVILLE, SC (US); MICHAEL TODD HARDMAN, VIRGINIA BEACH, VA (US); RICHARD TEWKSBURY, JR., LANSDALE, PA (US)

(21) Appl. No.: 17/978,329

(22) Filed: Nov. 1, 2022

Related U.S. Application Data
(60) Provisional application No. 63/276,047, filed on Nov. 5, 2021.

(51) Int. Cl. G16H 20/00; G16H 10/20; G06F 3/04817; G06F 3/04847

(52) U.S. Cl. CPC G16H 20/00; G06F 3/04817; G06F 3/04847; G16H 10/20

(57) ABSTRACT

The disclosure deals with a system and method for digitized data user-input device and method. In one exemplary embodiment, improved system and method are described for measuring user-controlled inputs. One exemplary such method relates to methodology for capturing, measuring, storing, analyzing, and reporting data corresponding to subjective experiences, particularly such as those of patients. The subject matter is particularly useful in the arena or field of healthcare generally, but can also be advantageously used for a wider setting of applications. A collection of images becomes representation of a continuum of images between polar images. As a controller is manipulated by a user, the images are displayed in direct relationship to the position of the slider. Only one image is visible at any time. The continuum of images may differ in step-wise progressions of shape, size, position of component elements, color, line density, rotation, skew, scale, or in other ways, such as opacity. The created collection of images creates in essence an animation, so that the progression of images are displayed, one at a time, in fixed sequence, beginning with one polarity and progressing through to the other polarity. Images in a given continuum (or collection of images) are assigned numerical values, e.g., from 1 - 21, or from 0 - 100, or otherwise, so as to represent scaled values. Likewise, the position of the control mechanism may be assigned scaled values, and the two may be correlated, so that the system determines a numerical input based on the user’s manipulation of images.

### PRIORITY CLAIM

The present application claims the benefit of priority of U.S. Provisional Pat. Application No. 63/276,047, titled DIGITIZED DATA USER-INPUT DEVICE AND METHOD, filed Nov. 5, 2021, and which is fully incorporated herein by reference for all purposes.

### BACKGROUND OF THE PRESENTLY DISCLOSED SUBJECT MATTER

The disclosure deals with a system and method for digitized data user-input. In one exemplary embodiment disclosed herewith, system and method for measuring user-controlled input is described. It is to be understood that while such exemplary embodiment is in the arena or field of healthcare generally, the presently disclosed subject matter can be used for a wider setting of applications.

The term “icons” in some instances herein refer to proprietary digitally-rendered GIFs or similar comprised of facial expressions and/or other percepts that are intended to represent subjective experiences including, but not necessarily limited to, emotions, feelings, affects, moods, sentiments, attitudes, judgments, and opinions, and which yield scalable data corresponding to those experiences.

Icons which are shape-shifting combine the attributes of affect displays and visual analogue scales in digital form, and they reflect contributions from the fields of cognitive science, neuropsychology, affect theory, computer science, developmental psychology, and others. More generically referred to as transformable or shape-shiftable objects, they are created and rendered via proprietary processes that provide for their generation, display, data production, storage, reporting, and data analysis. One example of such technology for measuring user-controlled input is provided in part by U.S. Pat. No. 9,959,011, the complete disclosure of which is incorporated herein by reference and for all purposes. However, such USP 9,959,011 requires use of an adjustable vector graphic, comprising a plurality of vector paths each including an anchor point and control points and being continuously adjustable between at least two differing predefined states. Such degree of complexity, relatively speaking, can require significant computational resources which can limit how robust a given application or use may be.

By comparison and contrast, presently disclosed subject matter makes use of a collection of images which can be relatively more efficiently handled and managed, resulting in relative advantages. For example, a relatively smaller required computing footprint can more readily make possible a mobile app version of the presently disclosed subject matter. The relatively created computational headroom also allows for the inclusion of other, added features such as custom imaging on the icons or other alternatives.

Thus, the presently disclosed subject matter offers advantages over inherent limitations posed by the above-referenced adjustable vector graphic technology.

### SUMMARY OF THE PRESENTLY DISCLOSED SUBJECT MATTER

The presently disclosed system and corresponding and/or associated methodology, broadly speaking, relates to improved technology for measuring user-controlled input.

It is to be understood that the presently disclosed subject matter equally relates to associated and/or corresponding methodologies. One exemplary such method relates to methodology for capturing, measuring, storing, analyzing, and reporting data corresponding to subjective experiences, particularly such as those of patients.

In some exemplary embodiments, a collection of images becomes representation of a continuum of images between polar images. As a controller is manipulated by a user, the images are displayed in direct relationship to the position of the slider. Only one image is visible at any time. The continuum of images may differ in step-wise progressions of shape, size, position of component elements, color, line density, rotation, skew, scale, or in other ways, such as opacity. The created collection of images creates in essence an animation, so that the progression of images are displayed, one at a time, in fixed sequence, beginning with one polarity and progressing through to the other polarity. Images in a given continuum (or collection of images) are assigned numerical values, e.g., from 1 - 21, or from 0 -100, or otherwise, so as to represent scaled values. Likewise, the position of the control mechanism may be assigned scaled values, and the two may be correlated, so that the system determines a numerical input based on the user’s manipulation of images.

One presently disclosed exemplary methodology preferably relates to methodology for predictively determining a patient’s likelihood to adhere to a healthcare treatment or activity plan for such patient. Such methodology preferably comprises creating a survey comprising a plurality of survey items related to selected determined obstacles to adherence; interactively conducting the survey for a given patient by having the patient use a movable feature of a digitized interface to respectively capture and definitively measure the patient’s subjective experiences for each of the plurality of survey items, to form a set of digitized data for the given patient for the corresponding plurality of survey items; and assessing the patient’s set of digitized data to determine a relative score for such patient for likelihood to adhere to a healthcare treatment or activity plan.

Another presently disclosed exemplary methodology preferably relates to methodology for predictively determining a participant’s likelihood to adhere to an activity plan for such participant. Such methodology preferably comprises interactively conducting a survey for a given participant by having the participant use a movable feature of a digitized interface to respectively capture and definitively measure the participant’s self-reported internal subjective feelings in response to a plurality of survey items concerning affect-based variables to likelihood of adherence, to form a set of internal self-reported data for the given participant; creating a database of measures of external fact-based data for the given participant; and collectively assessing the participant’s internal self-reported data and external fact-based data to determine a relative score for such participant for likelihood to adhere to an activity plan.

It is to be understood from the complete disclosure herewith that the presently disclosed subject matter equally relates to both methodology and corresponding and related apparatus.

Yet another presently disclosed exemplary embodiment preferably relates to a system for predictively determining a given patient’s likelihood to adhere to a healthcare treatment plan for such patient. Such system preferably comprises a memory; a display; and a processor coupled to the memory programmed with executable instructions. Such instructions preferably include a patient survey comprising a plurality of survey items to be administered to a given patient and related to selected determined obstacles to adherence to a healthcare treatment plan for such patient; a patient digitized interface comprising for each survey item a respective set of a collection of digitized images representative of a continuum of images between polar images for such patient to view on said display, and to capture and definitively measure subjective experiences thereof by such patient by having such patient use a movable feature to manipulate the appearance of each respective set of a collection of digitized images through a range of appearances thereof so that it reflects the self-reported intensity of how such patient feels in response to each item of the patient survey; and an assessing component, for assessing such patient’s set of self-reported responses to determine a relative score for such patient for likelihood to adhere to a healthcare treatment plan.

Other example aspects of the present disclosure are directed to systems, apparatus, tangible, non-transitory computer-readable media, user interfaces, memory devices, and electronic devices for improved data capturing and processing. To implement methodology and technology herewith, one or more processors may be provided, programmed to perform the steps and functions as called for by the presently disclosed subject matter, as will be understood by those of ordinary skill in the art.

Additional objects and advantages of the presently disclosed subject matter are set forth in, or will be apparent to, those of ordinary skill in the art from the detailed description herein. Also, it should be further appreciated that modifications and variations to the specifically illustrated, referred and discussed features, elements, and steps hereof may be practiced in various embodiments, uses, and practices of the presently disclosed subject matter without departing from the spirit and scope of the subject matter. Variations may include, but are not limited to, substitution of equivalent means, features, or steps for those illustrated, referenced, or discussed, and the functional, operational, or positional reversal of various parts, features, steps, or the like.

Still further, it is to be understood that different embodiments, as well as different presently preferred embodiments, of the presently disclosed subject matter may include various combinations or configurations of presently disclosed features, steps, or elements, or their equivalents (including combinations of features, parts, or steps or configurations thereof not expressly shown in the figures or stated in the detailed description of such figures). Additional embodiments of the presently disclosed subject matter, not necessarily expressed in the summarized section, may include and incorporate various combinations of aspects of features, components, or steps referenced in the summarized objects above, and/or other features, components, or steps as otherwise discussed in this application. Those of ordinary skill in the art will better appreciate the features and aspects of such embodiments, and others, upon review of the remainder of the specification, and will appreciate that the presently disclosed subject matter applies equally to corresponding methodologies as associated with practice of any of the present exemplary devices, and vice versa.

These and other features, aspects and advantages of various embodiments will become better understood with reference to the following description and appended claims. The accompanying drawings, which are incorporated in and constitute a part of this specification, illustrate embodiments of the present disclosure and, together with the description, serve to explain the related principles.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1A illustrates two exemplary factors (Stress, Anxiety) under assessment using presently disclosed subject matter implementing user-controlled slide bar inputs;

FIG. 1B illustrates the two exemplary factors (Stress, Anxiety) under assessment in present FIG. 1A, with exemplary user-controlled slide bar inputs moved to different exemplary positions, resulting in corresponding change of imagery;

FIG. 2A illustrates an exemplary factor (Loneliness) under assessment using presently disclosed subject matter implementing a user-controlled slide bar input;

FIG. 2B illustrates the exemplary factor (Loneliness) under assessment in present FIG. 2A, with exemplary user-controlled slide bar input moved to a different exemplary position, resulting in a corresponding change of imagery;

FIG. 3A illustrates three exemplary factors (Irritability, Depression, Sick/Healthy) under assessment using presently disclosed subject matter implementing user-controlled slide bar inputs;

FIG. 3B illustrates the three exemplary factors (Irritability, Depression, Sick/Healthy) under assessment in present FIG. 3A, with exemplary user-controlled slide bar inputs moved to different exemplary positions, resulting in corresponding change of imagery;

FIG. 4A illustrates two exemplary factors (Pain, Energy) under assessment using presently disclosed subject matter implementing user-controlled slide bar inputs;

FIG. 4B illustrates the two exemplary factors (Pain, Energy) under assessment in present FIG. 4A, with exemplary user-controlled slide bar inputs moved to different exemplary positions, resulting in corresponding change of imagery;

FIG. 5 illustrates an exemplary user-selected input for addressing an inquiry and indicating completion of survey in accordance with presently disclosed subject matter;

FIG. 6A illustrates exemplary demographic comparison data usable by an exemplary embodiment of presently disclosed subject matter;

FIG. 6B illustrates exemplary composite score feedback as produced by an exemplary embodiment of presently disclosed subject matter;

FIG. 7A represents exemplary factor scoring details as produced for the “STRESS” factor by an exemplary embodiment of presently disclosed subject matter;

FIG. 7B represents additional exemplary factor scorings as produced by an exemplary embodiment of presently disclosed subject matter;

FIG. 7C represents exemplary factor scoring details as produced for the “ANXIETY” factor by an exemplary embodiment of presently disclosed subject matter;

FIG. 7D represents exemplary factor scoring details as produced for the “IRRITABILITY” factor by an exemplary embodiment of presently disclosed subject matter; and

FIG. 7E illustrates notification to a user that user-input data has been augmented by additional data resources, in accordance with an exemplary embodiment of presently disclosed subject matter.

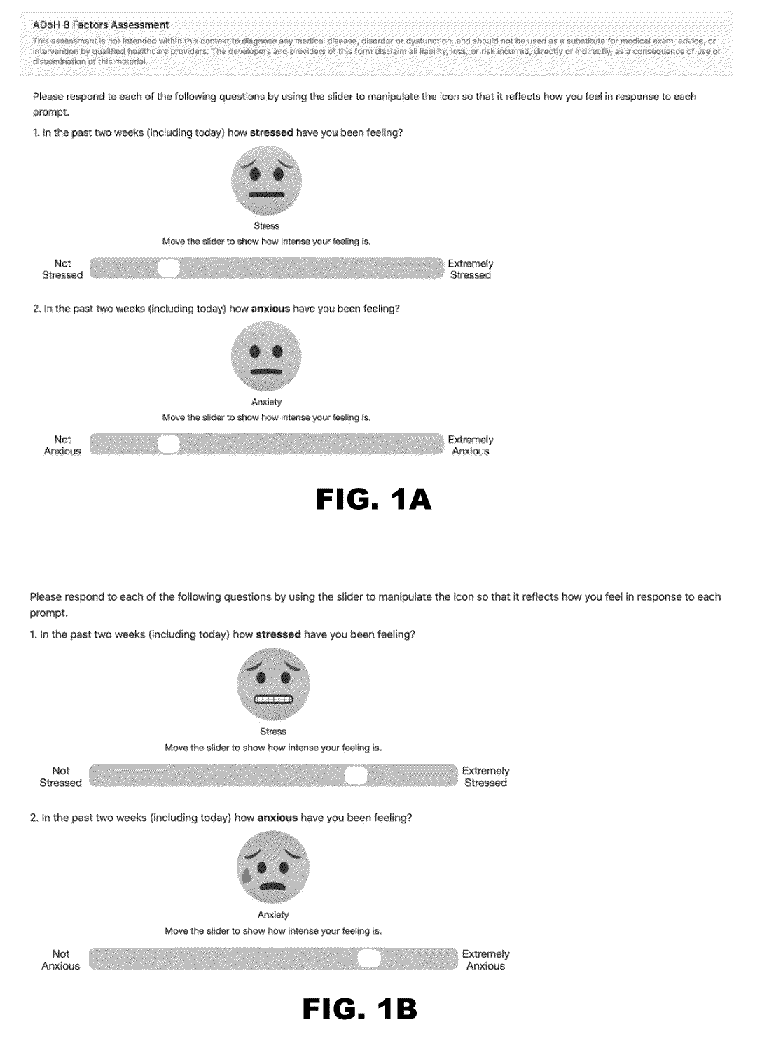

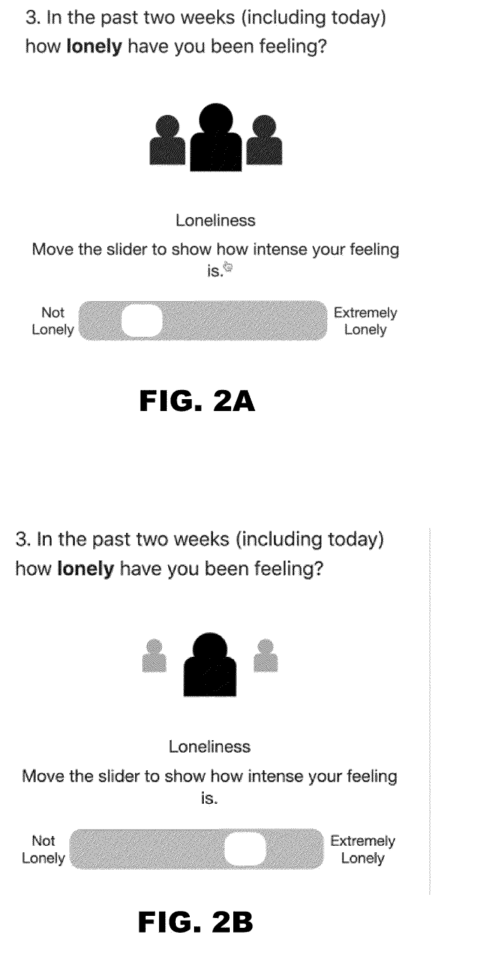

Repeat use of reference characters, Figure Nos. or descriptions in the present specification and drawings is intended to represent the same or analogous features, elements, or steps of the presently disclosed subject matter.

### DETAILED DESCRIPTION OF THE PRESENTLY DISCLOSED SUBJECT MATTER

It is to be understood by one of ordinary skill in the art that the present disclosure is a description of exemplary embodiments only, and is not intended as limiting the broader aspects of the disclosed subject matter. Each example is provided by way of explanation of the presently disclosed subject matter, not limitation of the presently disclosed subject matter. In fact, it will be apparent to those skilled in the art that various modifications and variations can be made in the presently disclosed subject matter without departing from the scope or spirit of the presently disclosed subject matter. For instance, features illustrated or described as part of one embodiment can be used with another embodiment to yield a still further embodiment. Thus, it is intended that the presently disclosed subject matter covers such modifications and variations as come within the scope of the appended claims and their equivalents.

In general, the present disclosure is directed to a system which, broadly speaking, relates to improved technology for measuring user-controlled input.

An exemplary embodiment of presently disclosed methodology may be implemented in the following manner. This particular embodiment uses an example comprising a bi-polar scale with a neutral setting between the polarities. Other variations of establishing ranges may be practiced.

As a first step (“step 1”), a practitioner of the presently disclosed subject matter may create three images. For example, the first image may represent “sick,” while the second image represents “neutral,” and the third image represents “well.”

As a next step (“step 2”), the practitioner may create one or more images representing equal-interval graded progressions between sick and neutral, and between neutral and well. This is the basis for a sequence of images that, broadly speaking, digitally or incrementally represent a continuum. Such continuum is thus comprised of a series of images representing discrete steps from one polarity to another.

In a most simplified embodiment, one might have as few as two images to represent a continuum. A more typical embodiment might have 5 or 7. In one exemplary presently preferred embodiment, 21 images may be used.

In such exemplary presently preferred embodiment, the collection of images has a super-ordinate label, such as “health” or “wellness.” This label represents a construct of clinical, health, healthcare, wellness, well-being, mental health, or similar relevance. In such exemplary embodiment, this label can be displayed to a user (person responding to a survey), but technically this is not necessarily required.

As a next further step (“step 3”), one may take such created collection of images and create in essence an animation of sort, so that the progression of images are displayed, one at a time, in fixed sequence, beginning with one polarity and progressing through to the other polarity. What a user sees is akin to what one sees while flipping through the pages of a “flip book.”

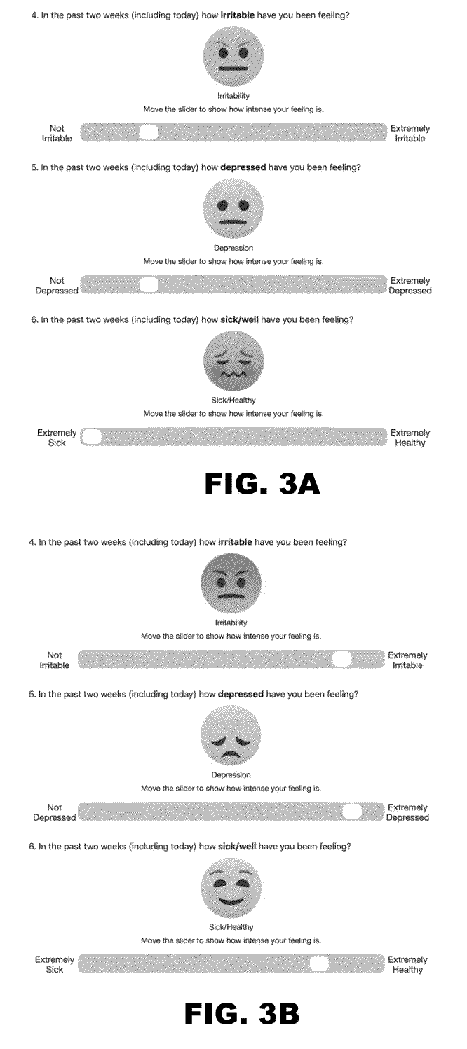

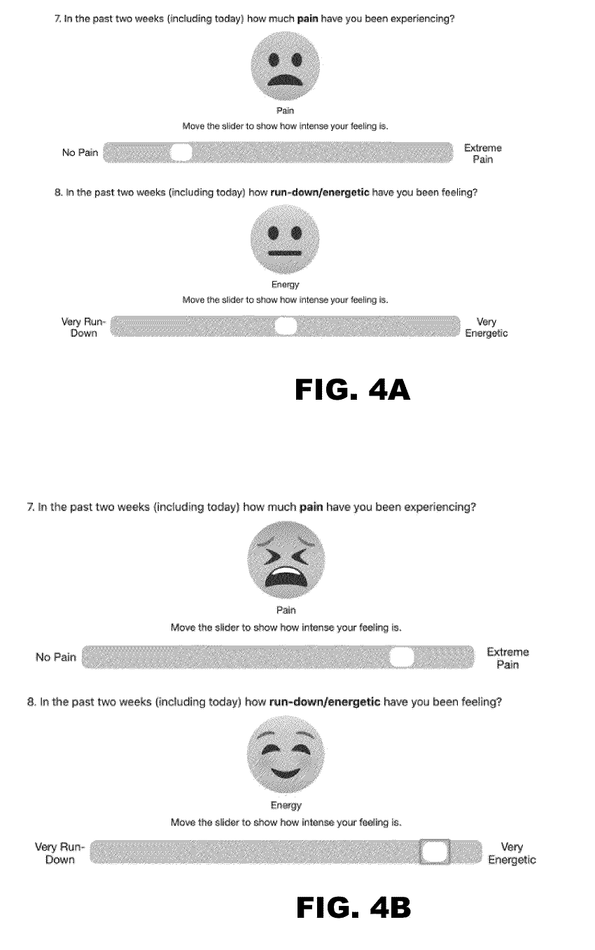

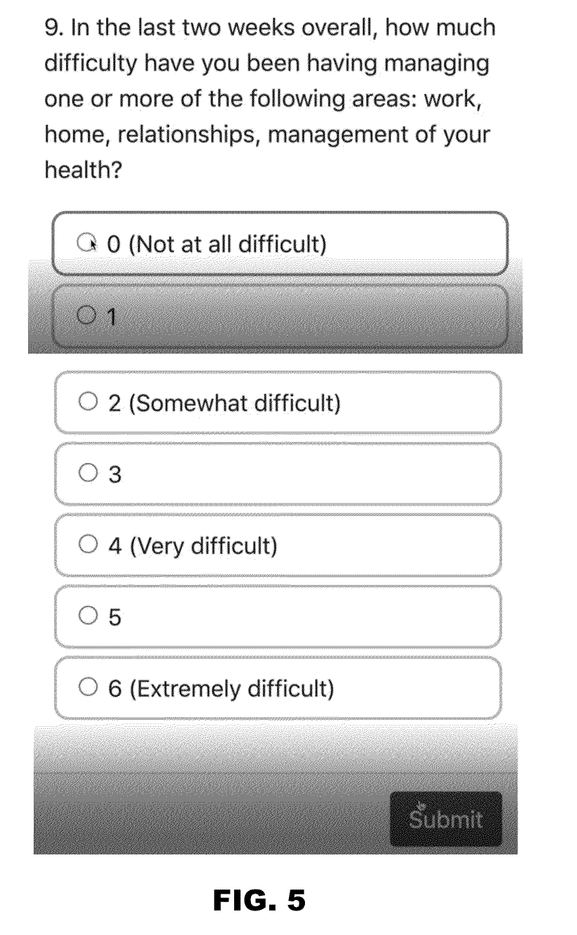

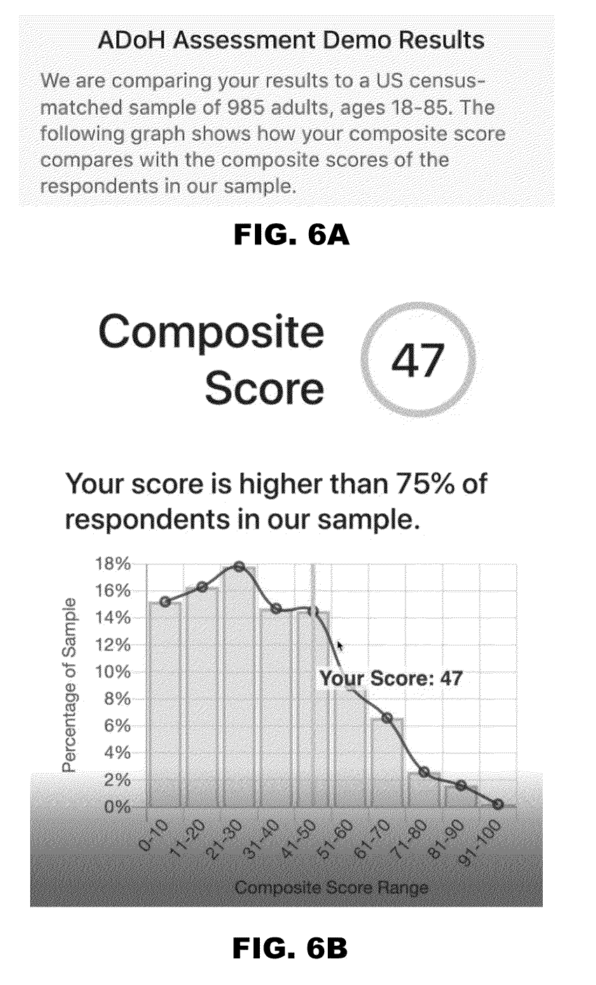

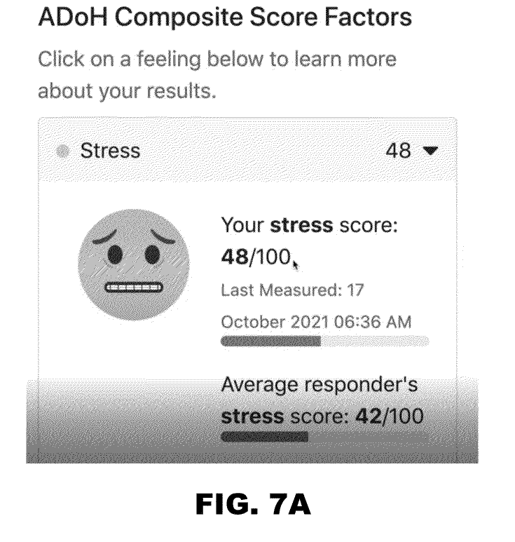

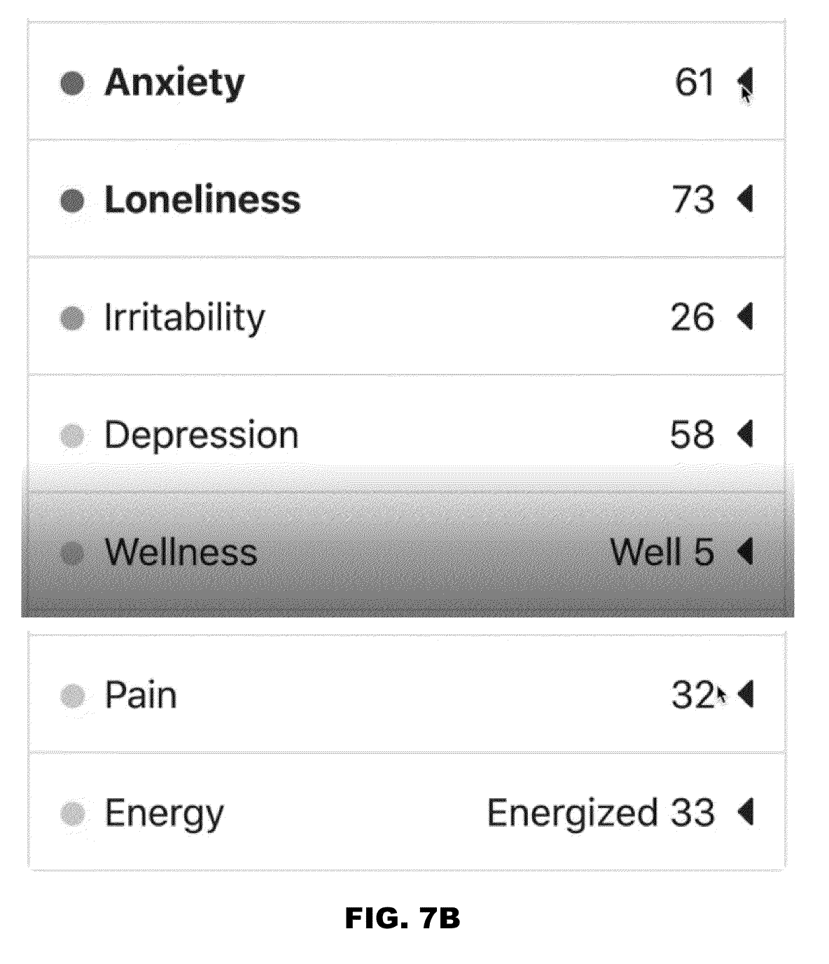

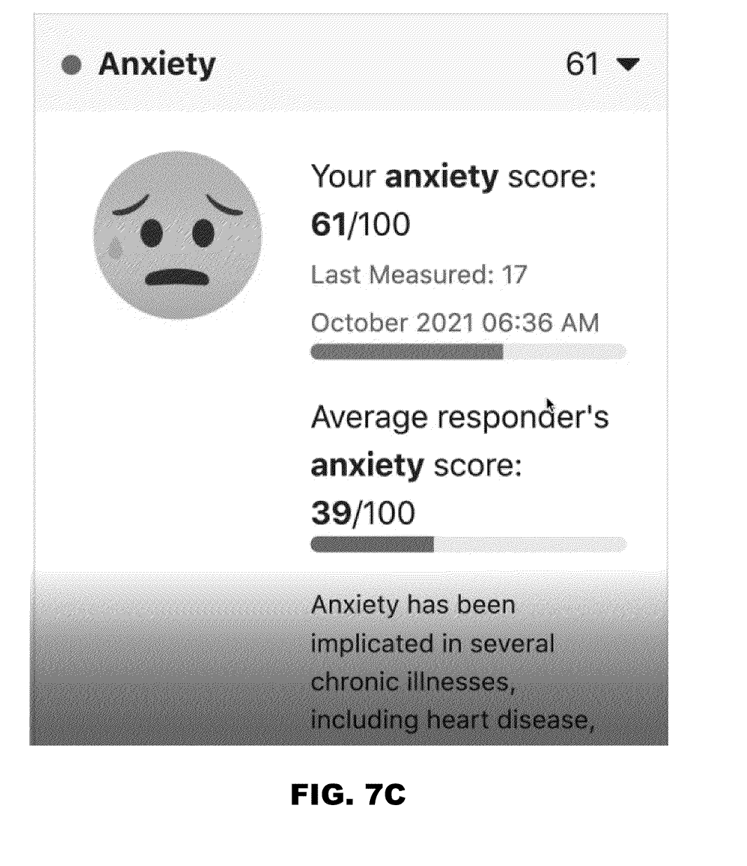

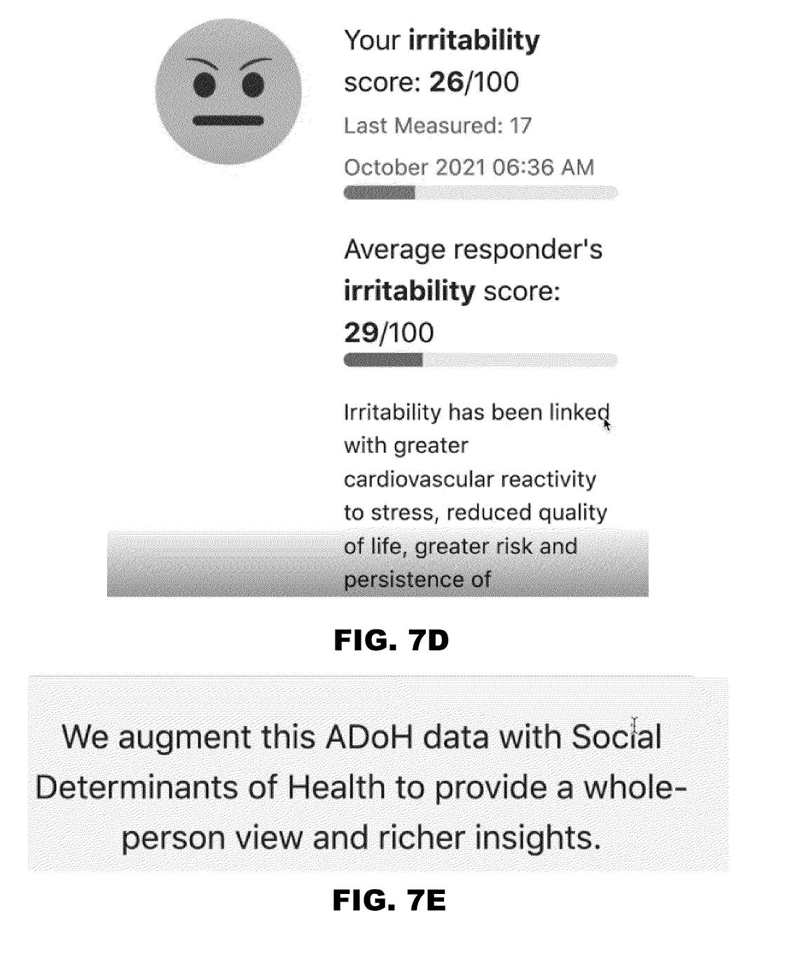

Per subsequent step (“step 4”), one may connect or associate a controller of some type (e.g., a slider, or a dial) to the animation. The controller allows the user to manually dictate the direction of the sequence of displays, the speed with which the images displace one another, and the subset of images that are displayed. For example, in FIGS. 3A and 3B, representative sliders are illustrated showing extremes going from “extremely sick” on one end to “extremely well” on the other. Thus, the sliders may be appended with text to label the polarities.

As the controller is manipulated, the images are displayed in direct relationship to the position of the slider. Only one image is visible at any time.

The continuum of images may differ in step-wise progressions of shape, size, position of component elements, color, line density, rotation, skew, scale, or in other ways, such as opacity.

In a next step (“step 5”), the images in a given continuum (or collection of images) are assigned numerical values, e.g., from 1 - 21, or from 0 - 100, or otherwise, so as to represent scaled values. Likewise, the position of the control mechanism may be assigned scaled values.

Per “step 6,” the scaled values of the images in progression are married to, so as to correspond with, the scaled values of the positions of the slider.

Per “step 7,” data are made available for storage, retrieval, and numerical operations. For example, the superordinate label “wellness” might be assigned a nominal value, such as Wellness = 1, so as to differentiate it in a database from another collection/continuum of images that collectively represent another construct (e.g., Depression = 2). The data representing each image in a continuum can be ordinal, interval or ratio type, thus representing scaled values.

Per “step 8,” the created data are stored in such manner as to be available for various operations in combination with other data from other sources.

Lastly, per “step 9,” the images of a continuum are displayed one at a time, controlled by a control mechanism such as a slider, and output can include numerical scaled scores for display to the user of the mechanism and/or to others who are consumers of the data. These displays may be of direct fidelity and/or of transformed values, such as might occur with composite score values from one or more collections from ≥ 1 constructs.

Certain exemplary technical aspects of exemplary implementation(s) may appear as follows:

A continuum may comprise a collection of GIFs, a lossless format of images that support and function both animated and static images. In one presently preferred exemplary embodiment, there are at least 21 GIFs for each scoring metric.

In general, a collection of GIFs for each metric are ordered in a continuous manner to represent at least a 100-point scoring system that represent equal-interval graded progression.

Such digitized form of continuous scoring metrics are able to be rendered distinctly for example in at least the following seven ways (other manners of rendering, now existing or later created, are intended to be included):
1. HTTP (hypertext transfer protocol).
2. Native iOS Software Development Kit (SDK).
3. Native Android Software Development Kit (SDK).
   (Derivatives of Android SDK Fire OS for Amazon Fire devices)
4. Executable Binary File (EXE) on Window PCS operating system environment.
5. ELF on Linux and Unix operating system environments.
6. Augmented Reality compatible programming languages (C ,C++, C#, C#-Unity, Java, JavaScript, Python, Swift).
7. Virtual Reality compatible programming languages (C, C++, C#, C#-Unity, Java, JavaScript, Python, Swift).

The presently disclosed subject matter equates in pertinent part to a scoring service. It has three ways it can be rendered and administered:
1. Over a web-service - for online instances.
2. Via an SDK or Exe - for offline instances.
3. Via a Natural User Input for AR and VR instances that can operate both online and offline.

The presently disclosed subject matter as a Service API is adaptable and configurable to:
1. Add scoring metric items.
2. Add validation for user input.
3. Add onboarding and how to instructions.
4. Render language and locale for instructions, onboarding and validations.

The presently disclosed scoring service or disclosed form of user-input has a number of distinct advantages. For example, there is increased efficiency because a third party server does not have to be accessed in order to create images. Also, the number of image files needed may be relatively reduced. In terms of implementation, the scoring service can be stress tested and load balanced now because it is implemented in a way which allows for monitoring, so that server pivot can be based on demand.

Thus, presently disclosed subject matter makes use of a collection of images which can be relatively more efficiently handled and managed, resulting in relative advantages. For example, a relatively smaller required computing footprint can more readily make possible a mobile app version of the presently disclosed subject matter. The relatively created computational headroom also allows for the inclusion of other, added features such as custom imaging on the icons or other alternatives.

Various improved features and functionalities of the presently disclosed subject matter may be described as follows. From a survey perspective, for example, digital format and electronic/computerized mechanisms may be utilized as opposed to paper. Presentation and display in digital media formats of one or more graphical images is achieved, variations of which can be displayed one in place of the other such that only one of a given image type and its variations are visible to a user at any given moment. At the same time, selection of a particular image may be controlled by a user by any of various means including, but not limited to, one or more control mechanical and/or digital mechanisms, one or more mechanical and/or digital dials, the tilt and/or position of a mobile device and modification of same via user manipulation of the device in two- and/or three-dimensional space, voice or other sound volume/pitch, manipulation of touch-capacitive screens or other input devices via movement of a finger or other body part along the screen in any of various directions including changes in direction and/or by tapping or other inputs, or similar.

Through practice of the presently disclosed subject matter, image(s) by design and/or by user perception/opinion represent one or more moods, emotions, feelings, and/or other subjective experiences (collectively also referenced herein as “feelings”). The variation(s) of said image(s) are intended and/or perceived to represent degrees of variation (i.e., intensity) of the feeling(s) being displayed, wherein the variation(s) of said image(s) may or may not be individually labelled in any particular way(s) to represent themselves specifically, or to represent themselves as variations of any particular image, or to represent themselves as any of various degrees of differentiation from one or more images in the collection of one or more images considered to be variations of one another, the collection of said images being labelled overtly or not with one or more words or symbols (e.g. in Chinese) that represent specific feeling(s), such that by means of user input the user can communicate the type and intensity of any of their various “feelings” via selection of one or more image types and manipulation of the variations of the image(s) displayed.

At the same time, per presently disclosed subject matter, such input of both type and intensity is represented/output also as data, to be defined as words, numeric symbols, or other symbols for the purpose of quantification of the type and/or intensity as appropriate to nominal, ordinal, interval, or ratio data types, such that the data output representations might or might not be visible to the user in pure form or in any of various translations, summaries, or other representations. Furthermore, such data output representations may be made available for storage in one or more databases and therefore at the present moment and/or at some later time via retrieval mechanisms made available for purposes including but not limited to mathematical operations on or with these data outputs within or beyond any particular storage and/or data manipulation mechanisms.

The relationship(s) between the user input method(s) and the image(s) and the variations of said images may be linear, logarithmic, or of other mathematical relationships; and the relationships may vary according to other variables and/or circumstances. The images can be varied as appropriate to particular present and/or future image creation/display technologies and/or formats. The image(s) and its/their variations can be configured and displayed so as to represent responses to one or more questions or other forms of prompts.

The data outputs of said image(s) and its/their variations can be configured and displayed so as to represent phenomena and/or constructs of clinical relevance, significance and/or utility, such as in (but not necessarily limited to) any of various measurement needs, desires, and/or demands in health, healthcare, wellness, well-being, wellbeing, performance/capacity, and related domains for means of description, explanation, prediction, prevention, modification, development, and/or cessation of attitudes, opinions, behaviors, emotions, moods, feelings, or other phenomena that may be and/or may become of interest to any of various providers, designers, supporters, and/or payers of products and/or services in these domains, and used singularly, in combination with one another, and/or in conjunction with other data types and/or sources.

This written description uses examples to disclose the presently disclosed subject matter, including the best mode, and also to enable any person skilled in the art to practice the presently disclosed subject matter, including making and using any devices or systems and performing any incorporated methods. The patentable scope of the presently disclosed subject matter is defined by the claims, and may include other examples that occur to those skilled in the art. Such other examples are intended to be within the scope of the claims if they include structural and/or step elements that do not differ from the literal language of the claims, or if they include equivalent structural and/or elements with insubstantial differences from the literal languages of the claims.

What is claimed is:

1. A methodology for predictively determining a patient’s likelihood to adhere to a healthcare treatment or activity plan for such patient, comprising:
creating a survey comprising a plurality of survey items related to selected determined obstacles to adherence;
interactively conducting the survey for a given patient by having the patient use a movable feature of a digitized interface to respectively capture and definitively measure the patient’s subjective experiences for each of the plurality of survey items, to form a set of digitized data for the given patient for the corresponding plurality of survey items; and
assessing the patient’s set of digitized data to determine a relative score for such patient for likelihood to adhere to a healthcare treatment or activity plan.

2. The methodology according to claim 1, wherein each survey item comprises a respective set of a collection of digitized images representative of a continuum of images between polar images, with the movable feature of the digitized interface manipulated by a user so that respective digitized images are displayed in direct relationship to the position of the movable feature.

3. The methodology according to claim 2, wherein:
a respective set of a collection of digitized images includes at least five respective images of the set; and
only one image of a respective set of a collection of digitized images is visible by the patient user at any one time.

4. The methodology according to claim 2, wherein:
the continuum of images differ in step-wise progressions of at least one of shape, size, position of component elements, color, line density, rotation, skew, scale, or opacity or other features thereof visible to a user; and
the patient-moved position of the movable feature of the digitized interface is assigned scaled values, which is correlated with a digitized image of a respective set of a collection of digitized images, for determining a numerical input based on the user’s manipulation of digitized images.

5. The methodology according to claim 2, wherein a respective set of a collection of digitized images for at least one of the survey items and a movable feature of a digitized interface for the patient to manipulate through a range of appearances are designed to correlate with the intensity of the patient’s feelings in response to a survey item, and the definitive measurement captured with each corresponds directly to the final point of manipulation established by the patient through such range of appearances in response to a particular survey item.

6. The methodology according to claim 5, wherein the respective set of a collection of digitized images are pre-validated for a selected population of patients in which the given patient is a member, to validate that each respective set of a collection of digitized images and its range of images represents the subject matter of its associated item and attendant range of intensities thereof.

7. The methodology according to claim 2, wherein assessing the patient’s set of data includes at least one of:
establishing a predictive score for the given patient indexed relative to ranges of scoring of sets of data for responses to survey items involving the same respective sets of collections of digitized images for the respective survey items as have been used in the survey for the given patient and pre-validated for a selected population of patients in which the given patient is a member, or
establishing a specific score within an indexed range to relatively assess the probability of adherence to a healthcare treatment or activity plan for the given patient.

8. The methodology according to claim 1, further including:
reporting the determined relative score to at least one of the given patient, healthcare staff supporting the patient, researchers, administrators, payors, and supportive associates of the given patient; and
subsequently interactively conducting the same or a different survey for the given patient at a later point in time, and reporting an updated relative score to at least one of the given patient, healthcare staff supporting the patient, researchers, administrators, payors, and supportive associates of the given patient.

9. The methodology according to claim 1, further including:
creating a database of measures of the patient’s health status indicators and external social and economic variables to likelihood of adherence, to form a set of fact-based data for the given participant; and
collectively assessing the patient’s internal self-reported data and fact-based data to determine a relative score for the given patient for likelihood to adhere to a healthcare treatment or activity plan for the given patient.

10. The methodology according to claim 9, wherein the external social and economic variables to likelihood of adherence for a patient comprise social/economic factors experienced by an individual patient that negatively impact medical treatment plan adherence, including at least one of low socioeconomic status, low health literacy, low levels of education, low levels of social support, unemployment, housing instability, family dysfunction, barriers with transportation to medical care, high medication costs, and negative cultural beliefs about medical treatment.

11. The methodology according to claim 1, wherein the survey items include at least one of a question, an image, a statement, a sound bite, and a video file presented to the patient for capture of the patient’s subjective response thereto.

12. The methodology according to claim 1, wherein the internal subjective feelings which are variables to likelihood of adherence for a patient include self-reported felt sense and intensity thereof for at least one of wellness versus illness, stress, depression, anxiety, pain, loneliness of the patient, and satisfaction with the patient’s most recent health provider/staff interaction.

13. A system for predictively determining a given patient’s likelihood to adhere to a healthcare treatment plan for such patient, comprising:
a memory;
a display; and
a processor coupled to the memory programmed with executable instructions, the instructions including:
a patient survey comprising a plurality of survey items to be administered to a given patient and related to selected determined obstacles to adherence to a healthcare treatment plan for such patient,
a patient digitized interface comprising for each survey item a respective set of a collection of digitized images representative of a continuum of images between polar images for such patient to view on said display, and to capture and definitively measure subjective experiences thereof by such patient by having such patient use a movable feature to manipulate the appearance of each respective set of a collection of digitized images through a range of appearances thereof so that it reflects the self-reported intensity of how such patient feels in response to each item of the patient survey, and
an assessing component, for assessing such patient’s set of self-reported responses to determine a relative score for such patient for likelihood to adhere to a healthcare treatment plan.

14. The system according to claim 13, wherein:
the system is implemented via a hardware and software platform comprising a plurality of network-based non-transitory storage devices, servers, and processors, which are accessible by authorized users; and
the system includes at least one network-based non-transitory storage device for at least one of:
being accessed by authorized users, for the update and storage therein of data on at least one particular patient concerning at least one of background external health, social, and economic variables to likelihood of adherence for such particular patient, or
being accessed by at least one particular patient, for the update and storage therein of data on at least one of definitively measured subjective experiences for such particular patient in response to at least one item of the patient survey.

15. The system according to claim 13, wherein the assessing component is further operative for at least one of:
collectively assessing such patient’s set of self-reported responses normalized relative to stored data of anonymized results from a plurality of other patients with common or similar background data,
storing on at least one network-based non-transitory storage device the self-reported responses and relative score for such patient for likelihood to adhere to a healthcare treatment plan, to be accessed by authorized users, or
storing on at least one network-based non-transitory storage device the self-reported responses and relative scores for such patient, based on repeated administrations of the same or a different patient survey to such patient, for likelihood to adhere to a healthcare treatment plan data for such patient, to be accessed by authorized users.

16. The system according to claim 13, wherein the respective sets of collections of digitized images are pre-validated for a selected population of patients in which the given patient is a member, to validate that each respective set of collection of digitized images and its range of appearances represents the subject matter of its associated item and attendant range of intensities thereof.

17. The system according to claim 13, wherein the continuum of images differ in step-wise progressions of at least one of shape, size, position of component elements, color, line density, rotation, skew, scale, or opacity or other features thereof visible to a user.

18. A methodology for predictively determining a participant’s likelihood to adhere to an activity plan for such participant, comprising:
interactively conducting a survey for a given participant by having the participant use a movable feature of a digitized interface to respectively capture and definitively measure the participant’s self-reported internal subjective feelings in response to a plurality of survey items concerning affect-based variables to likelihood of adherence, to form a set of internal self-reported data for the given participant;
creating a database of measures of external fact-based data for the given participant; and
collectively assessing the participant’s internal self-reported data and external fact-based data to determine a relative score for such participant for likelihood to adhere to an activity plan.

19. The methodology according to claim 18, wherein each survey item comprises a respective set of a collection of digitized images representative of a continuum of images between polar images, with the controller manipulated by a participant so that respective digitized images are displayed in direct relationship to the position of the slider.

20. The methodology according to claim 18, wherein:
the affect-based variables comprise determined obstacles to achievement of activities in an activity plan; and
the participant’s set of external fact-based data comprises external social and economic variables to likelihood of adherence for the given participant.

21. The methodology according to claim 19, wherein:
the respective set of a collection of digitized images for each respective survey item are pre-validated for a population of participants in which the given participant is a member, to validate that each respective set of a collection of digitized images represents the subject matter of its associated item and attendant range of intensities thereof;
the survey items include at least one of a question, an image, a statement, a sound bite, and a video file presented to the participant for capture of the participant’s subjective response thereto; and
collectively assessing the participant’s internal self-reported data and external fact-based data includes establishing a specific score within an indexed range to relatively assess the probability for such participant for likelihood to adhere to an activity plan.

22. The methodology according to claim 18, wherein the participant comprises one of:
(a) a potential consumer for a given product, and the survey items relate to a particular product or service of potential interest to the potential consumer, as part of evaluating customer experiences or conducting consumer research relative to such particular product or service, and
(b) a patient and the activity plan comprises a health treatment plan for the patient.

23. The methodology according to claim 18, wherein:
the participant comprises a patient and the activity plan comprises a health treatment plan for the patient; and
the methodology further includes subsequently interactively conducting the same or a different survey for the given patient at a later point in time, and reporting an updated relative score to at least one of the given patient, healthcare staff supporting the patient, researchers, administrators, payors, and supportive associates of the given patient.
